(19) United States
(12) Patent Application Publication
(10) Pub. No.: US 2022/0110786 A1
(43) Pub. Date: Apr. 14, 2022

(54) DEFORMABLE THERMAL PACK

(71) Applicant: NYCE INNOVATIONS, LLC., Canton, MA (US)

(72) Inventor: Robert BASCHNAGEL, Garden City, NY (US)

(21) Appl. No.: 17/556,353

(22) Filed: Dec. 20, 2021

Related U.S. Application Data
(63) Continuation-in-part of application No. 16/512,782, filed on Jul. 16, 2019, now Pat. No. 11,003,897.
Application No. 16/512,782 is a division of application No. 16/379,221, filed on Apr. 9, 2019, now Pat. No. 11,179,271.
Application No. 16/379,221 is a continuation-in-part of application No. 16/034,388, filed on Jul. 13, 2018, now Pat. No. 11,192,696.
(60) Provisional application No. 62/604,611, filed on Jul. 14, 2017.

(51) Int. Cl. A61F 7/02

(52) U.S. Cl. CPC A61F 7/02; A61F 2007/0039; A61F 2007/023

(57) ABSTRACT

A thermal pack including: a flexible pouch body having a plurality of pouch segments each defining a cavity; and one or more hook and loop straps attached to a first end of the pouch body, where the pouch body has an opening for accepting a second end of the hook and loop strap for securing the pouch body to a body part. The thermal pack can also include a bendable and deformable support frame having at least two twisted wires on at least two longer sides of the pouch body and/or metallic cubes disposed in each of the cavities of the pouch segments, each of the metallic cubes having a metallic shell defining a cavity with a freezable substance.

### CROSS REFERENCES TO RELATED APPLICATIONS

The present application is a Continuation-In-Part Application of U.S. application Ser. No. 16/512,782 filed on Oct. 28, 2021, which is a Divisional Application of U.S. application Ser. No. 16/379,221 filed on Apr. 9, 2019, which is a Continuation-In-Part Application of U.S. application Ser. No. 16/034,388 filed on Jul. 13, 2018, which claims priority to provisional application 62/604,611 filed on Jul. 14, 2017, by the present inventor and entitled “A Thermal Treatment Pack.”

### BACKGROUND

### Field

The present disclosure relates generally to treatment by applied temperature controlled materials, and, in particular, relates the treatment of pain or soreness, and, in greater particularity, relates to thermal treatment of pain or soreness by applied hot or cold packs.

Numerous types of ice packs and heat packs use straps for holding the packs to the body.

Further, many devices are shown in issued patents and patent applications publications such as the following: US Patent Application Pub. 2001/0051820 shows thermal treatment packs and the retainers for such. These are highly configurable with the use of cloth strips with fasteners. US Patent Application Pub. 2004/0167456 shows a medical wrap about an ankle using hook and loop. Ice or heat packs may be placed thereunder. US Patent Application Pub. 2004/0244412 shows a pouch for ice bags that is attached to clothing by safety pins. US Patent Application Pub. 2001/0051820 shows a pouch with adhesive tabs for attachment. US Patent Application Pub. 2006/0004427 shows a cylindrical tubular body of flexible material with a temperature retaining material therein that can be used on an arm or leg, but not a shoulder. US Patent Application Pub. 2006/0081000 shows a self-adhesive ice bag device pouch. US Patent Application Pub. 2008/0140166 shows a thermal pack with modules inside. One version is tubular. US Patent Application Pub. 2009/0205106 shows a tubular wrist band with elastic bands on the edges, but it does not provide for heating or cooling but for absorbing water on the wrist. US Patent Application Pub. 2011/0093050 shows a thermal wrap with a pouch for foot use. The wrap is held in place by Velcro straps. U.S. Pat. No. 5,395,399 shows a thermal wrap with a pouch for thermal material and is held in place by an elastic cloth. U.S. Pat. No. 7,060,086 shows a tubular thermal pack for use on hands and legs. U.S. Pat. No. 8,603,151 shows a cooling device for application to body parts having an external thermal source and is held in place by straps. All of these references are incorporated by reference.

Thus, there is a need for a more convenient device to threat pain or soreness.

### SUMMARY

The present embodiments provide a flexible device for providing a source of thermal energy for treating pain or soreness in an arm, leg or shoulder.

A thermal pack may be applied to the body on an arm or leg or shoulder. A pouch for holding a thermal pack or being the thermal pack itself is held between parallel deformable, bendable rods that remain in that position to which bent until removed or adjusted. The rods may be on all four sides. The pack is generally rectangularly shaped with rounded corners, but other shapes may be considered. One end may be open and sealed by Velcro® after the appropriate thermal source whether a cooling source or heating source is placed therein, but the pouch may also hold a hot/cold therapy solution. The pouch may be an ice pack. If the source is the pouch, it is sold as a unit. The pack may come in various sizes for an arm, leg or shoulder. Additional features may be added to the pouch such as handles to aid in applying the pack. In general, the pack is held against the body part, i.e., wrist, and then bent around the wrist forming a “loosely” fitting cuff. This would be also done on the shoulder since the bendable rods will remain bent. The embodiments are directed at a person normally at rest and not contemplated for a person actively moving.

In another embodiment, a gel pack being either a cold pack or a heat pack, is fixedly attached to the flexible rods and may be used also to keep foods warm or cold, for example.

It is an object to provide a pack using thermal energy to treat pain or soreness;

It is another object to provide a pack being either hot or cold in this treatment;

It is a further object to provide a flexible thermal pack that loosely grips the body part and remains there while the body remains inactive;

It is still a further object to provide thermal pack whose shape is controlled by flexible and deformable rods;

It is still another object to provide a thermal treatment pack that does not require the use of straps;

It is still another object to provide hot/cold therapy packs for active people such as baby boomers or elderly who have a limited range of motion, or for people who are out of shape where the bendable rods may be tightened for greater compression.

It is still another object to provide a thermal treatment pack that is self-contained that can be temperature adjusted by cooling or heating the whole pack.

These and other objects, features, and advantages of the present invention will become more readily apparent from the attached drawings and the detailed description of the preferred embodiments, which follow.

### BRIEF DESCRIPTION OF THE DRAWINGS

FIG. 1 is a perspective view of a thermal treatment pack formed into a cuff;

FIG. 2 is a perspective view showing the thermal treatment pack before the insertion of the flexible rods therein and before the insertion of a thermal source in a pouch;

FIG. 3 is a perspective view of the thermal treatment pack as the flexible rods are being inserted of the present invention;

FIG. 4 shows the thermal treatment pack before the insertion of the heat source of the present invention;

FIG. 5 shows the heat source being inserted into the pouch of the thermal treatment pack;

FIG. 6 shows the heat source within the pouch of FIG. 5;

FIG. 7 shows the thermal treatment pack formed into a cuff and placed about a wrist;

FIG. 8 shows the thermal treatment source with the heat source integrated into the pouch and further having handles thereon attached to the flexible rods

FIG. 9 shows alternative handles on the heat thermal treatment pack;

FIG. 10 shows the thermal treatment pack of FIG. 8 applied to a lower leg section with the handles gripping the leg;

FIG. 11 shows the thermal treatment pack of FIG. 8 applied to a wrist by use of straps in the handles;

FIG. 12 shows partially a twisted wire as the frame with a foam cover supporting the thermal source;

FIG. 13 shows a strap secured about the handles of the thermal treatment pack like that of FIG. 11 applied to a wrist;

FIG. 14 shows a thermal treatment pack with the thermal source secured by bonding to the flexible rods thereabout;

FIG. 15 shows the thermal source having edges for bonding to the flexible rods thereabout;

FIG. 16 shows thermal sources being gel packs having edge for bonding to the flexible rods thereabout;

FIG. 17A shows by top view the thermal treatment pack; FIG. 17B shows a side view of the pack of FIG. 17A; FIG. 17C shows the thermal source with edge brackets bonded to the flexible rods thereabout with corner connectors for the flexible rods;

FIG. 18A show the pack being bent and before placement in a container such as a cooler; and FIG. 18B shows the bent pack inside of a container;

FIG. 19 illustrates a configuration of two thermal packs connected together and used on an ankle region of a user;

FIG. 20 illustrates a thermal pack from the two thermal packs illustrated in FIG. 1;

FIG. 21 illustrates an embodiment of an elongated bendable member for use in the thermal packs;

FIG. 22 illustrates a cup and ball rod component of the elongated bendable member of FIG. 21;

FIG. 23 illustrates a thermal pack employing the elongated bendable member of FIG. 21;

FIG. 24 illustrates another embodiment of a thermal pack;

FIG. 25 illustrates a perspective view of the thermal pack of FIG. 24;

FIG. 26 illustrates a sectional view of the thermal pack of FIG. 25 as taken along line 26-26 in FIG. 25;

FIG. 27 illustrates a configuration of two thermal packs connected together;

FIG. 28 illustrates the two thermal packs of FIG. 27 used on a shoulder region of a user;

FIG. 29 illustrates another embodiment of a thermal pack having a removable bendable elongated wire;

FIG. 30 illustrates another embodiment of a thermal pack having adhesion portions at ends thereof;

FIG. 31 illustrates the thermal pack of FIG. 30 used on a head of an infant.

FIG. 32 illustrates another embodiment of a thermal pack having a slot for removing/inserting a secondary pouch having a thermal material;

FIG. 33 illustrates the thermal pack of FIG. 32 in which the thermal material is being removed from the slot;

FIG. 34 illustrates a thermal pack having an embodiment of support frame;

FIG. 35 illustrates a portion of the support frame of FIG. 34;

FIG. 36 illustrates a sectional view of the support frame of FIG. 35 as taken along line 36-36 in FIG. 35;

FIGS. 37-39 illustrate another embodiment of a thermal pack;

FIGS. 40-42 illustrate a variation of the thermal pack of FIGS. 37-39;

FIGS. 43-45 illustrate another variation of the thermal pack of FIGS. 37-39;

FIGS. 46-48 illustrate a variation of the thermal packs of FIGS. 37-39, 40-42 and 43-45, respectively;

FIG. 49 illustrates a use of the thermal packs of FIGS. 37-48;

FIG. 50 illustrates another use of the thermal packs of FIGS. 37-48;

FIGS. 51A and 51B illustrate the thermal pack of FIG. 50;

FIGS. 52A and 52B illustrate the thermal pack of FIGS. 46-48; and

FIGS. 53A and 53B illustrate first and second alternative uses for any of the thermal packs of FIGS. 37-52B.

Like reference numerals refer to like parts throughout the several views of the drawings.

### DETAILED DESCRIPTION

In general, a thermal treatment pack is applied to the body on an arm or leg or shoulder. A pouch for holding a thermal pack or being the thermal pack itself is held between parallel deformable, bendable rods that remain in that position to which bent. The rods may be on all four sides. The pack is generally rectangularly shaped with rounded corners, but other shapes may be considered. One end may be open and sealed by Velcro® after the appropriate thermal source whether a cooling source or heating source, but the pouch may also hold a hot/cold therapy solution. The pouch may be an ice pack. If the source is the pouch, it is sold as a unit. The pack may come in various sizes for an arm, leg or shoulder. Additional features may be added to the pouch such as handles to aid in applying the pack. In general, the pack is held against the body part, i.e., wrist, and then bent around the wrist forming a “loosely” fitting cuff. This would be also done on the shoulder since the bendable rods will remain bent. The invention is directed at a person normally at rest and not contemplated for a person actively moving. In another embodiment of the present invention, a gel pack being either a cold pack or a heat pack, is fixedly attached to the flexible rod and may be used also to keep foods warm or cold.

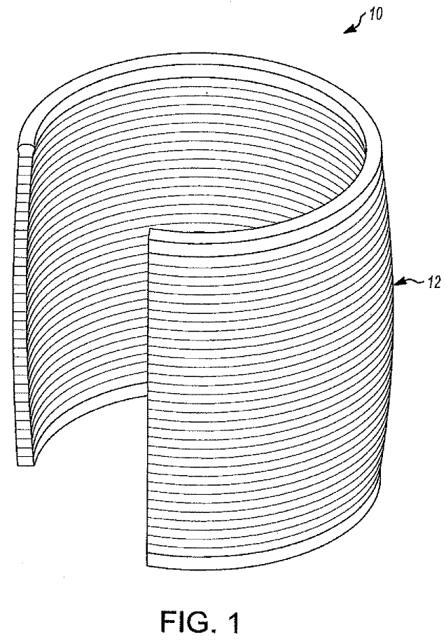

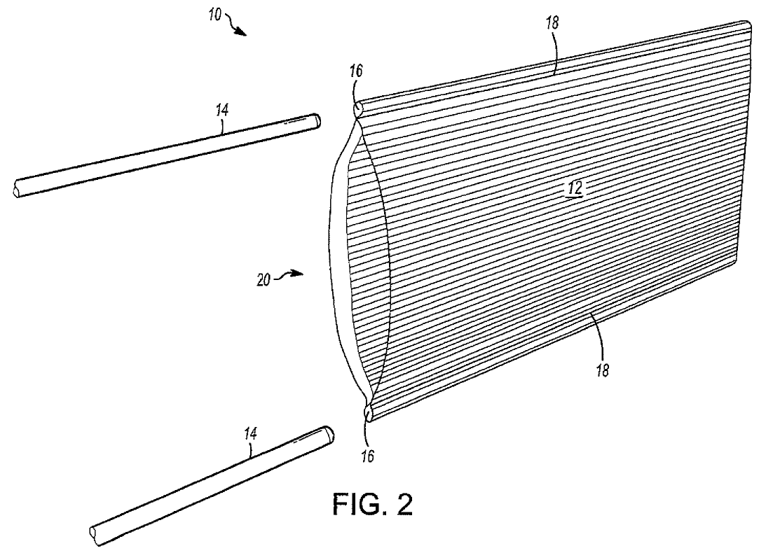

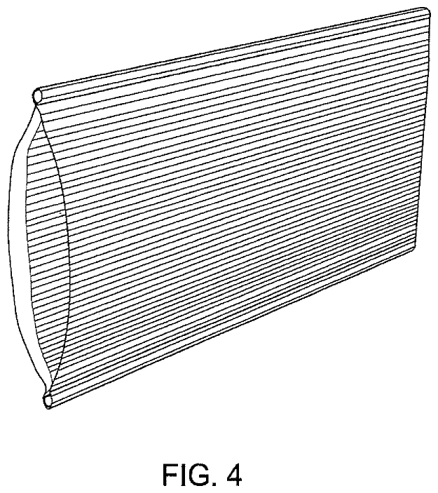

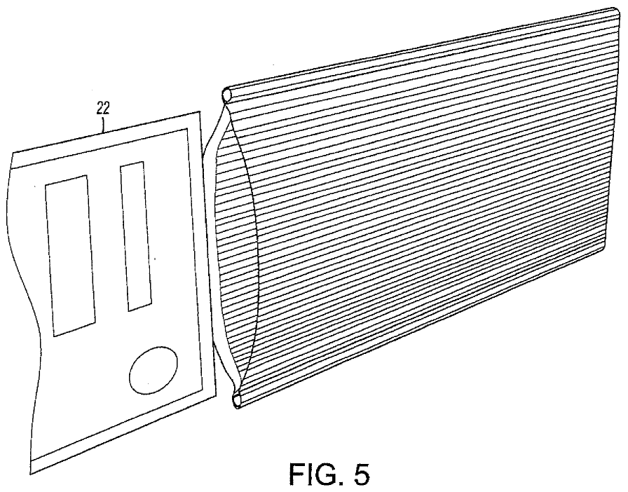

FIG. 1 shows a deformable thermal treatment pack 10 that can be applied to the body such as at a leg, arm and even a shoulder with a thermal source or pouch 12 therein. The pack 10 is bent like a cuff and will remain in that shape since deformable rods are placed thereabout. See FIGS. 7, 10 and 11. In FIG. 2, the pouch 12 is held between two parallel deformable, bendable rods 14 that are enclosed in cylindrical sleeves 16 as shown in FIGS. 2 to 6. These rods provide a support frame for the pouch. The bendable rods 14 form a support frame 17 about the pouch 12. The sleeves 16 are made by stitching away from the pouch edges 18 an appropriate distance inward. Other conventional means such as gluing or heat bonding can be used to form the sleeves depending on the materials. The pack 10 is generally rectangularly shaped but other shapes may be used. It may be about 2 to 4 inches on a short edge and 4 to 6 inches on a long edge. One end 20 may be open and sealed by Velcro™ after the appropriate thermal source 22, FIG. 5, whether a cooling source or heating source is inserted into the pouch 10.

In another embodiment, the appropriate thermal source whether an ice pack or hot/cold therapy solution may already be sealed in the pouch and sold as a unit. The walls of the pack 10 may be water impermeable then. The pack 10 may come in various sizes for an arm, leg or shoulder. FIG. 7, to be applied, the pack 10 is held against the body part, i.e., wrist 24, and then bent around the wrist 24 forming a cuff such as shown in FIG. 7. This would be also done on the shoulder and since the bendable rods will remain bent, the bent pack 10 will loosely grip the shoulder, but additional compression may be used or available. The pack 10 may tightly enclose a portion of the body part, and thus be sufficiently in contact with the skin to provide appropriate thermal treatment. The use of straps would not be required. Although the pack 10 is best used on inactive body parts, it can be also used in active movement. The bendable rods may be on all four sides. The corner devices of the pack may be rounded. This product may be ideal for baby boomers, athletes, elderly with limited motion, and out-of-shape customers.

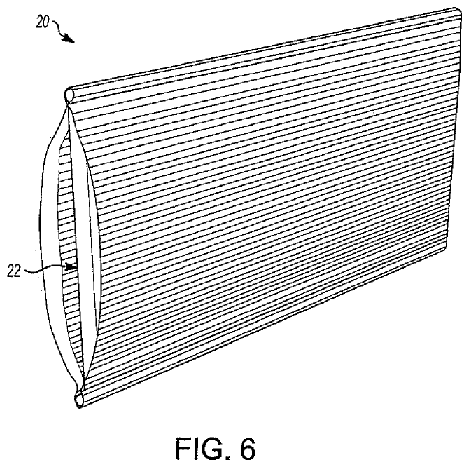

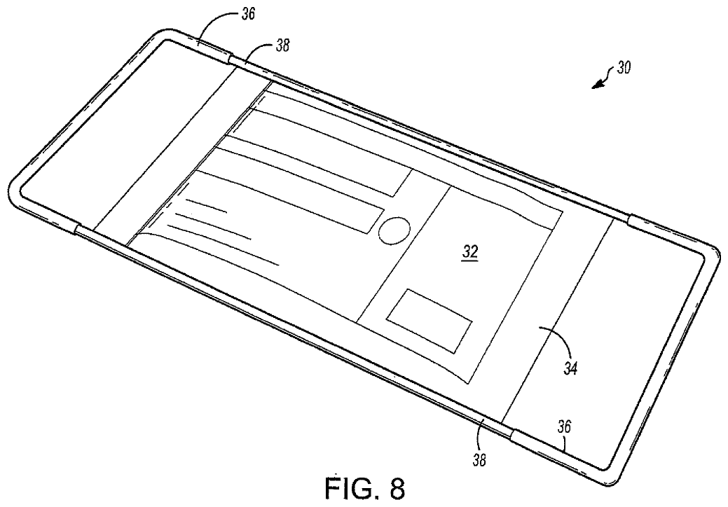

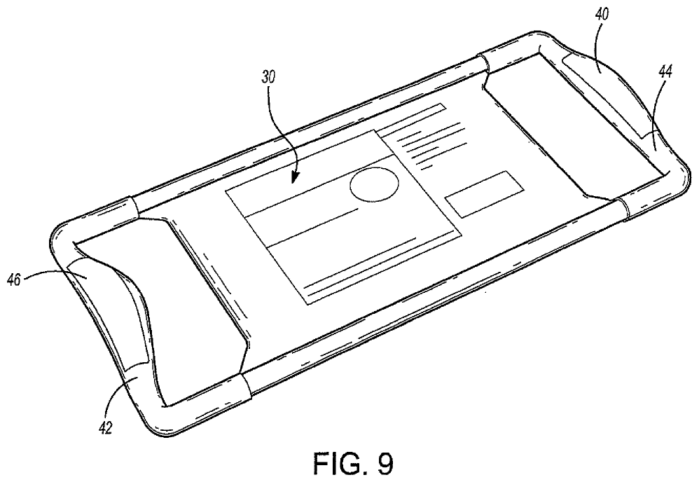

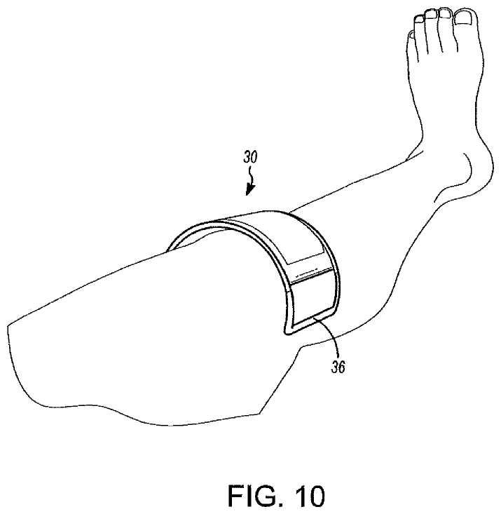

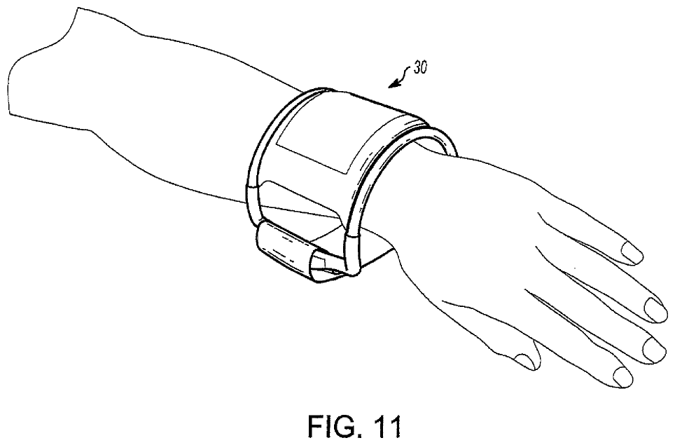

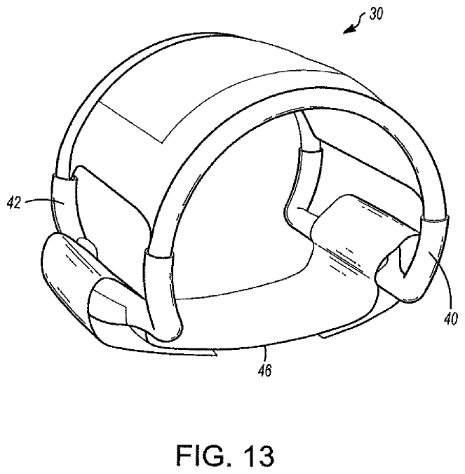

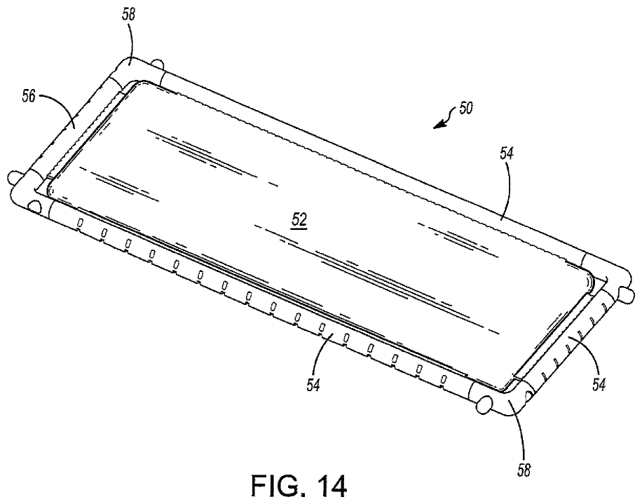

In another embodiment, FIG. 8 shows a thermal pack 30 with a thermal source 32 integrated into a pouch 34 and further having handles 36 thereon attached to the flexible, deformable and bendable rods 38 that provide a support frame. The handles 36 may be removed from the bendable rods 38, but can otherwise aid in the placement of the pack 30. The thermal source 32/pouch 30 is rectangular in shape and made of plastic-like material which would be water-proof. The thermal source 32 may be reusable by placing in a refrigerator or a microwave or a one-time use of heating or cooling material therein. The handles 36 provide a further reach about a body part that may have an irregular shape and may also be made of flexible, bendable rods like used in tie-wraps. The handles 36 shown are U-shaped. See FIGS. 14 to 17. FIG. 10 shows the thermal treatment pack 30 of FIG. 8 applied to a lower leg section, and FIG. 11 shows the thermal treatment pack 30 of FIG. 8 applied to a wrist.

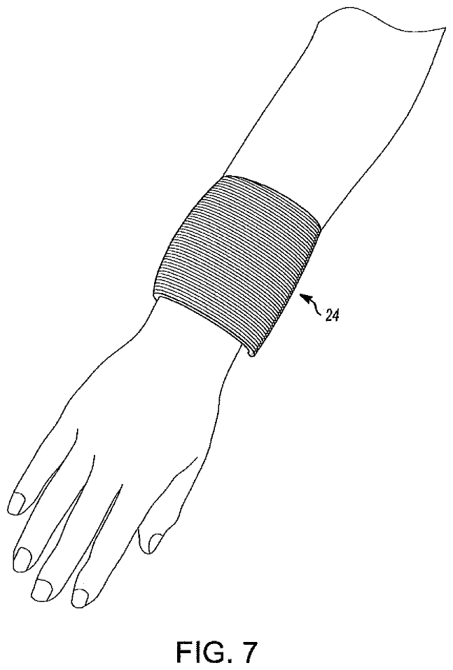

FIG. 9 shows alternative handles 40 and 42 on the thermal treatment pack 30. The alternative handles 40 and 42 may have flattened sections 44 and 46, either being oriented perpendicular or parallel to the pouch. This provides for ease of handling and also for attachment of a strap 46 as shown in FIGS. 11, 12, and 13. With the use of a strap 46, the pack 30 can even be used on an active person.

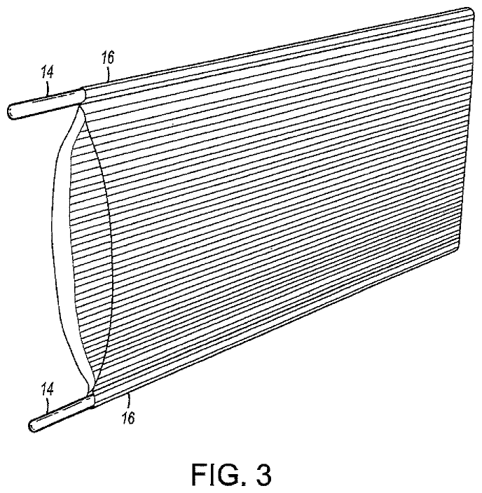

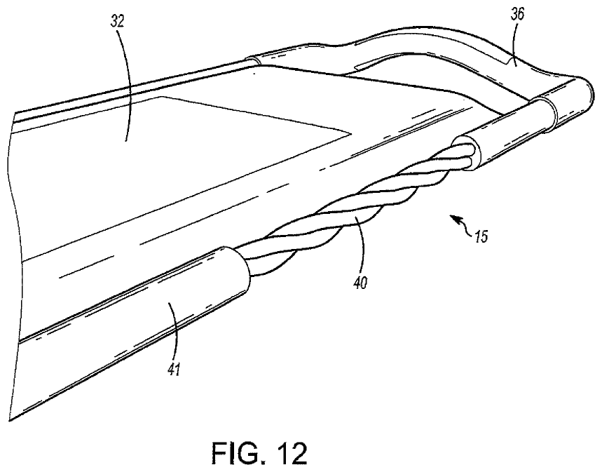

Referring to FIGS. 2 and 3, the bendable or flexible or deformable rods 14 are like tie wraps. They are flexible, i.e., can be bent to a particular shape and will remain in that shape until bent further, and will resist bending to a certain degree. They may be made of a foam rod thereabout with the twisted wires therein. See FIG. 12. These bendable rods 38 may be of tubular shape as shown. The bendable rods 41, in FIG. 12, may be twisted wires 40 inside of a tube 41.

Referring to FIGS. 14 to 17, in FIG. 14, a thermal treatment pack 50 is shown. A pouch 52 is designed to hold either cooling gel or heating gel or other material such as ice. The pouch 52 is plastic and is permanently and fixedly attached to two flexible side rods 54 being made of a foam material and having therein wires, not shown. Further, two end rods 56 are also flexible if used. The side rods 54 and end rods 56 may be jointed together by corner support devices 58 that are typically rounded.

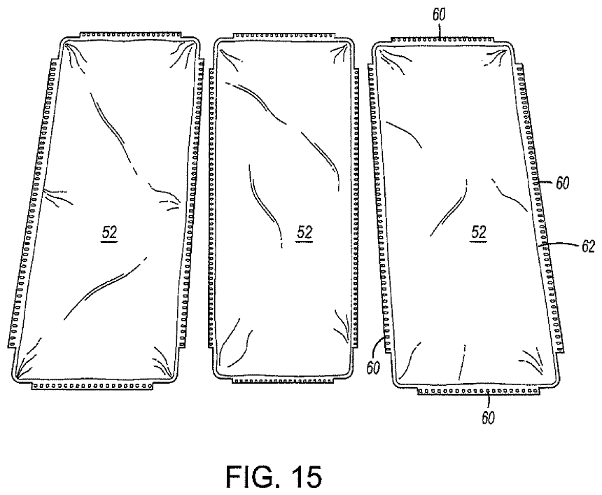

Referring to FIG. 15, the pouch 52 has extended edges 60 being made of similar material as the pouch 52. The pouch 52 may be about 3 to 4 inches wide and 6 inches long. Other sizes may be used since it may be used on a leg having a larger diameter than a wrist. The extended edges 60 extend therefrom by about ¼ to ½ inch and has a plurality of slotted holes 62 running along the length. In the embodiment of FIG. 14, both the sides and the ends of the pouch have this extended edge 60 thereon.

These extended edges 60 used a vulcanization process to secure the edges 60 to the gel pack 52. The bendable rods 54/56 were enclosed in a foam and then secured to the edges 60 by ultrasonic welding. The pouches 52/60 have the extended edges of either the sides or the ends or both. The foam rods with corners are placed about the pouches 52/60. The pouch 52 shown in FIG. 14, being white, can hold material that maybe activated to provide heat. Referring to FIG. 16, a pouch 66 shown may be blue in color and filled with cold gel that may be placed in a refrigerator for cooling. Similar edges 60 having a slotted pattern thereon allow for secure attachment to the foam rods by vulcanization.

To cover a large area, the pack 50 may be attached to similar packs 50 by use of snap fittings as shown in FIG. 14. Each side of the pack 50 has a male snap fitting 90 and a female snap fitting 92. The opposite snap fittings are attached to each corner devices 58 and when constructed the other adjacent side has the snap fittings reversed so that one pack 50 may be pushed into an adjacent pack 50.

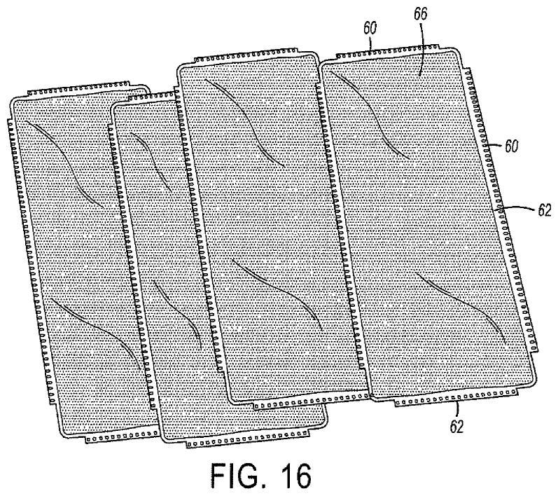

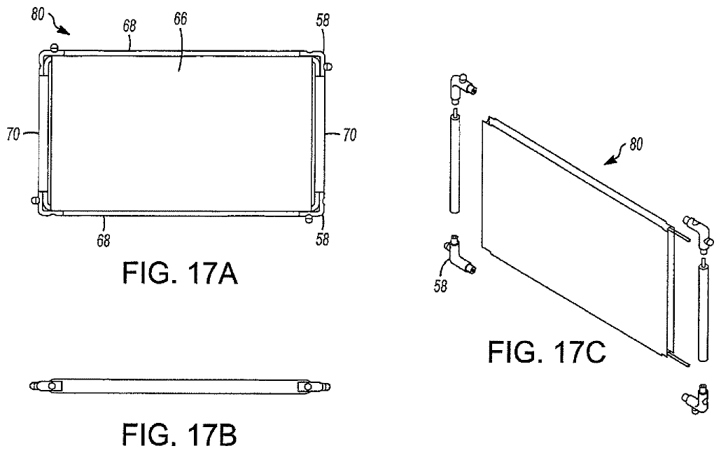

FIGS. 17A, 17B and 17C illustrate a pack 80 assembled and disassembled showing the various items thereon.

In a further embodiment as shown in FIGS. 18A and 18B, the thermal pack 61 if a cold pouch with gel material or ice therein, may be used in food storage and allows for molding or shaping of the pack 61 to more closely fit about food items, not shown, in a container 62.

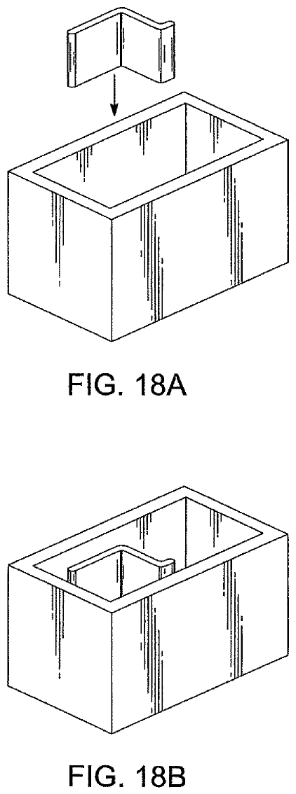

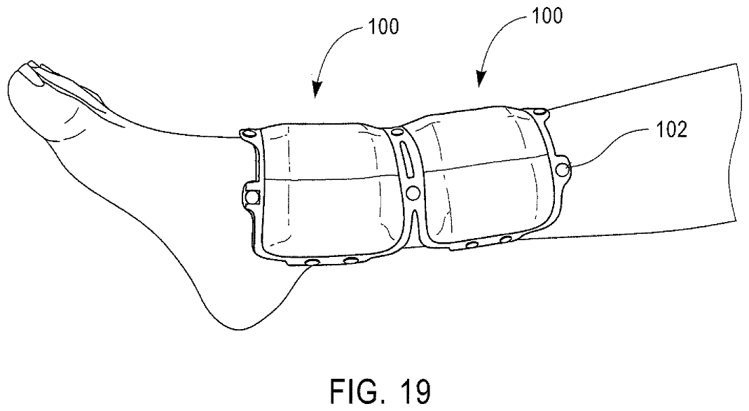

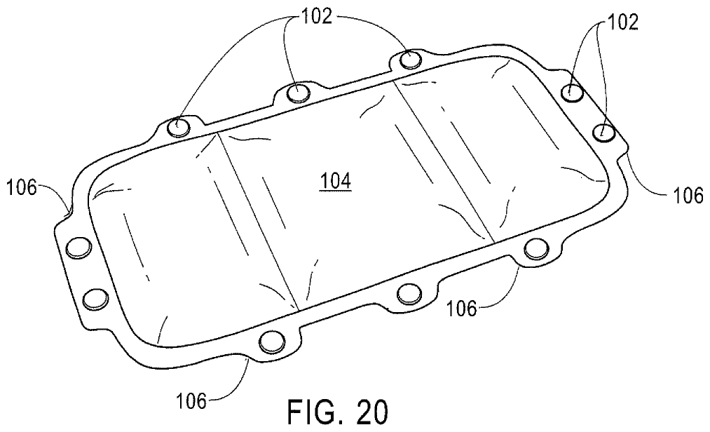

Referring now to FIGS. 19 and 20, there is shown a thermal pack as described above. FIG. 20 shows one such thermal pack 100, while FIG. 19 shows two thermal packs 100 attached together with connectors, such as snaps 102, where the two thermal packs 100 are formed to conform to a shape of an ankle area of a user. The snaps 102 can be configured as those known in the art, such as those used on canvas boat covers, having a male part and a female part which mate together when the male part is pressed into the female part and which can be separated with the application of a force separating the male part from the female part. FIGS. 19 and 20 illustrate the snaps 102 being spaced longitudinally on each of the four sides of the thermal pack 100. The snaps 102 can be providing on only one side, two sides or three sides and can be spaced in any intervals and provided in any number, including a single snap 102. The snaps 102 can be disposed on a pouch projection 106 in a one-to-one correspondence or two or more-to-one correspondence. The snaps 102 can be formed of metal or plastic. Plastic snaps being capable of being used in a microwave where the thermal material in the pouch is being heated in a microwave.

The thermal pack 100 includes a pouch 104 containing a thermal source in a cavity formed by two sides of the pouch 104. The thermal source can be either heated or cooled to apply either hot or cold, respectively, to the ankle area or other body area. Some materials are capable of being both heated and cooled, thereby allowing the thermal pack 100 to be used for application of both heat and cold to a body part of the user. The pouch 104 is formed of materials, such as neoprene and canvas. While all portions of the pouch 104 can be the same material, more than one material can be used to form the pouch 104. For example, one side of the pouch 104 can be a first material, such as neoprene and another side of the pouch 104 can be a second material, such as canvas.

A bendable and deformable support (not shown in FIGS. 19 and 20) that is either attached to or disposed within the pouch 104 is provided such that the pouch 104 can be deformed into a shape matching a contour of a body part and the pouch is maintained in the shape while applied to the body part by the bendable and deformable support frame. Although the bendable and deformable support frame can be provided on all sides of the thermal pack, the bendable and deformable support frame can be providing on only a single side or only on two opposing sides. Still further, the bendable and deformable support frame can be providing interior to the pouch. The bendable and deformable support frame can be a single or plurality of twisted wires formed of a metal material having the desired properties of being capable of being deformed into a shape matching a contour of a body part and allowing the pouch to be maintained in the shape while applied to the body part by the bendable and deformable support frame. The wire or plurality of twisted wires can be encased in an elastomer/resin casing so as to be safe to microwave in the case where the bendable and deformable support frame is fixed to the pouch and the thermal material is heated in the microwave.

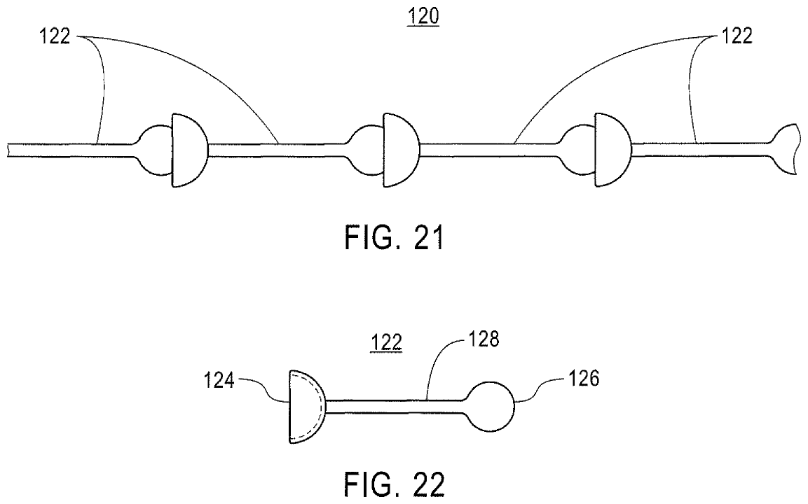

FIGS. 21 and 22 illustrate another embodiment of the bendable support frame in the form of an elongated member 120 having the same properties as a wire in that the elongated member 120 is capable of being deformed into a shape matching a contour of a body part and allowing the pouch to be maintained in the shape while applied to the body part. The elongated member 120 comprises a series of cup and ball members 122. Each cup and ball member 122 includes a cup 124 and ball 126 at each end of a shaft 128. The ball 126 of each cup and ball member 122 fits into a cup 124 of an adjacent cup and ball member 122 such that the ball 126 rotates in the cup 124 in a way to achieve the desired properties discussed above. The ball and cup members 122 can be formed of a resin material and the elongated member 120 can be further covered with a covering, such as a heat shrink tubing, to enhance such properties.

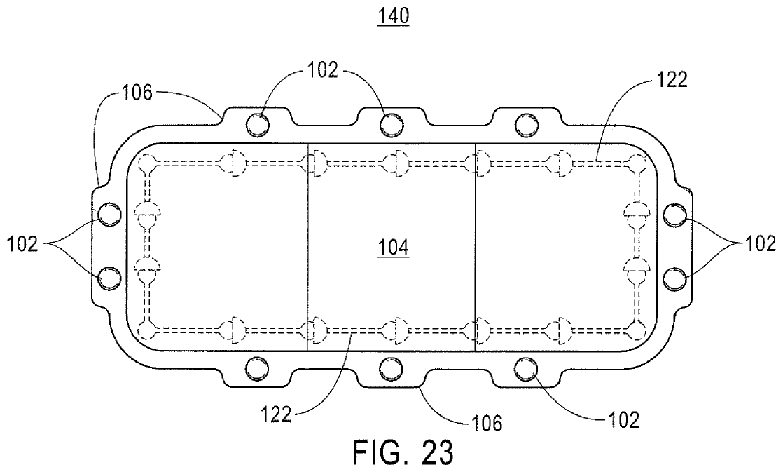

FIG. 23 illustrates an embodiment of thermal pack 140 similar to that of FIG. 20 with the exception that the elongated member 122 is disposed on all four sides of the pouch 104 and within the pouch 104. Alternatively, the elongated member 122 can be provided on less than all four sides and in an interior of the thermal pouch 104. Furthermore, the elongated member 122 can be disposed along an edge of the thermal pack 140 outside of a cavity formed in the pouch 104. Furthermore, the elongated member 122 can be removable from the pouch 104 or thermal pack 140.

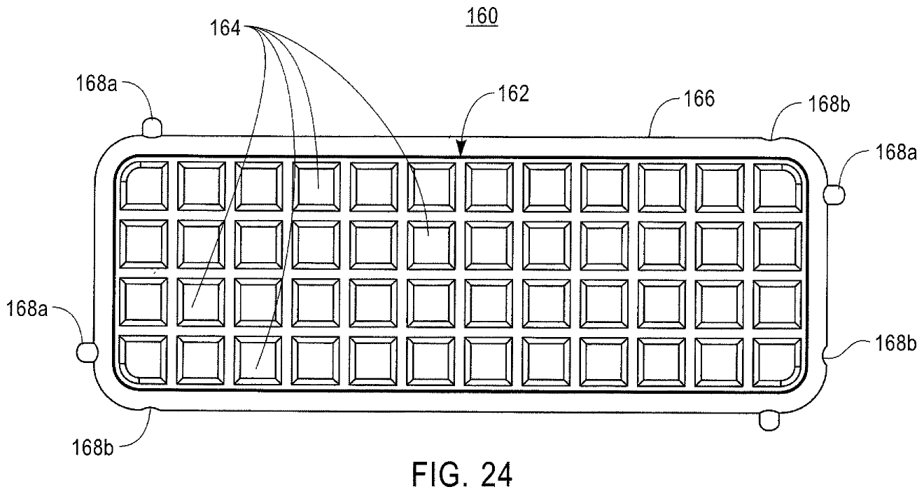

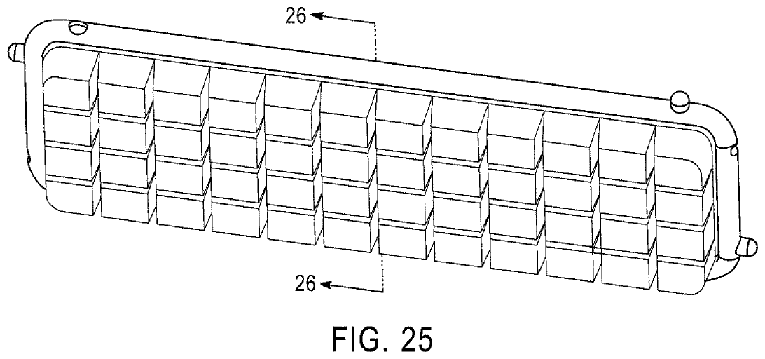

Referring now to FIGS. 24-26, there is shown another embodiment of a thermal pack 160 having a segmented pouch 162 formed of a plurality of pouch segments 164. The pouch segments 164 can be any shape or size and provided in any number greater than two. The thermal pack 160 of FIG. 24 illustrates a great number of small pouch segments 164 for increasing flexibility of the pouch and concentrating an amount of thermal material contained in each pouch segment 164.

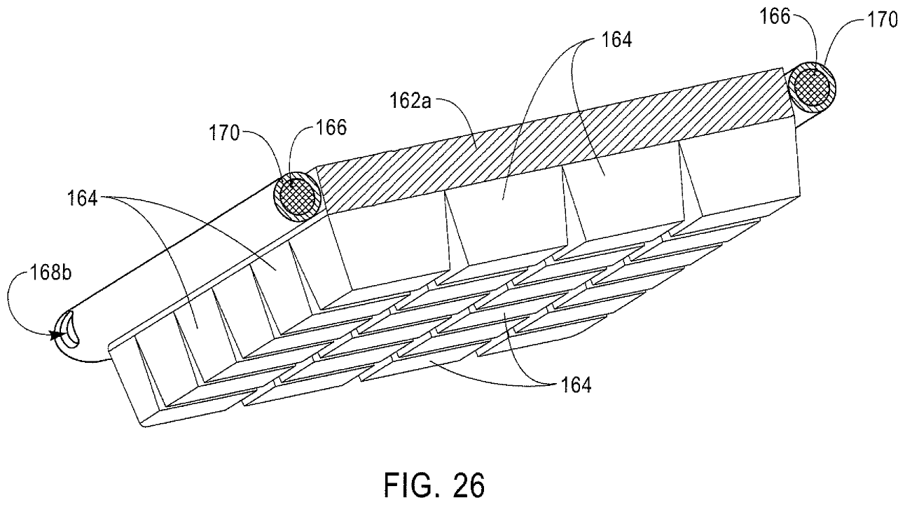

The thermal pack 160 also includes a bendable and deformable support frame 166 provided at all four sides of the pouch 162 to outline the plurality of pouch segments 164. Male and female connectors 168a, 168b are also provided on a periphery of the thermal pack to allow similarly configured thermal packs to be connected together with the thermal pack 160. As shown in FIG. 26, the pouch 162 is formed having the thermal material in both a common portion 162a of the pouch 162 and in each of the pouch segments 164. Furthermore, the bendable and deformable support frame 166 is provided in a cavity 170 apart from the cavity forming the pouch 162.

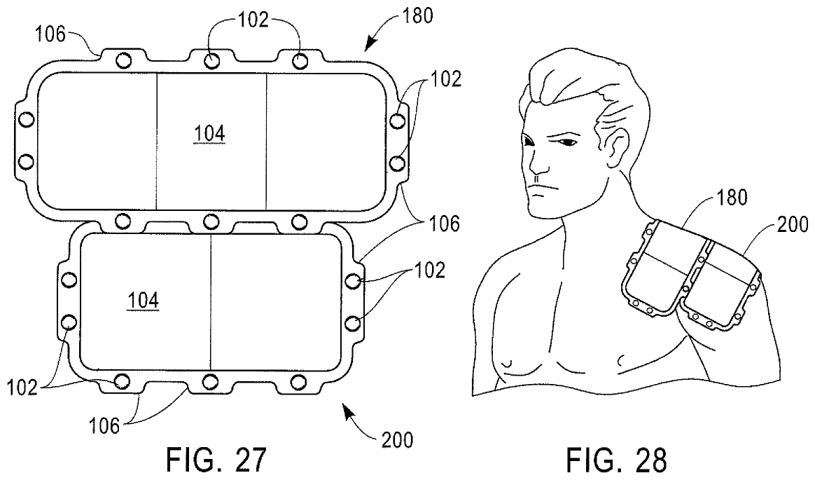

Referring now to FIGS. 27 and 28, there are illustrated first and second thermal packs 180, 200 each having similar features to those described above with regard to any of FIGS. 20, 23 and 25. Thermal pack 180, although larger in size than thermal pack 200 has a similar connector (snaps 102) configuration and spacing such that the larger thermal pack 180 can be connected to the smaller thermal pack 200 to conform with irregular body surfaces, such as the shoulder area illustrated in FIG. 28.

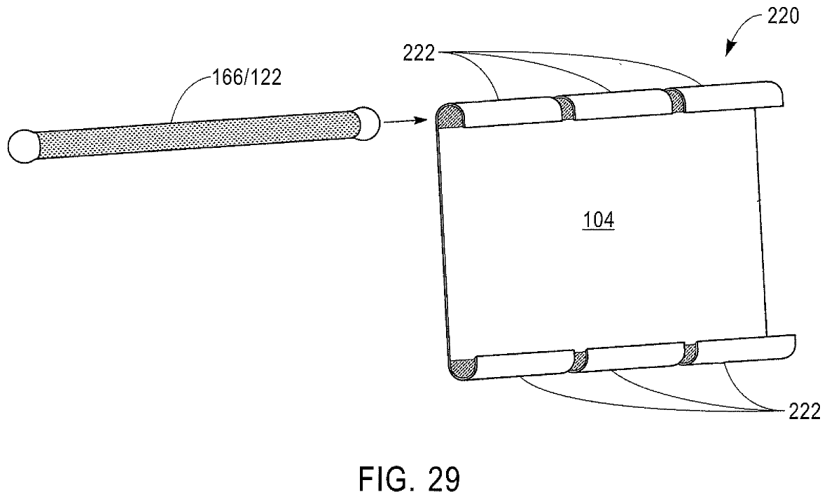

Referring now to FIG. 29, there is shown schematically a thermal pack 220 having similar features to those described above with regard to any of FIGS. 20, 23 and 25. However, in the thermal pack 220 of FIG. 29, the bendable and deformable support frame 166 is removable from the pouch 104. In such configuration, the pouch has ends 222 which can secure the bendable and deformable support frame 166 to the pouch 104. Although a means of releasable securing known in the art can be employed to attach the bendable and deformable support frame 166 to the pouch 104, in the embodiment of FIG. 29, the bendable and deformable support frame 166 has one of a hook or loop fastener (e.g., Velcro™) and the pouch ends 222 has the other of the hook or loop fastener. Such configuration allows use of differently configured bendable and deformable support frame 166 and also allows use of metal components for the bendable and deformable support frame 166 which can be removed when microwaving where the thermal material in the pouch 104 is being heated.

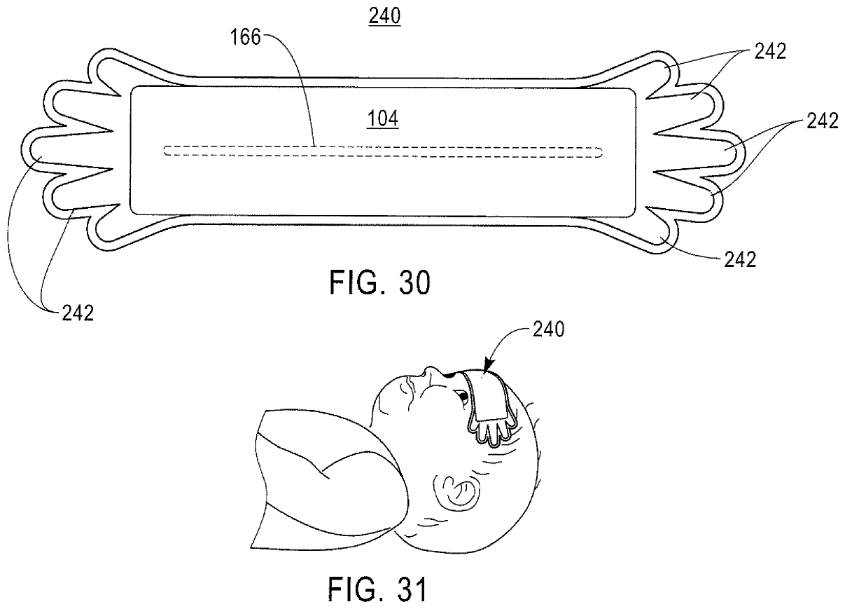

Turning next to FIGS. 30 and 31, there is shown another embodiment of thermal pack 240. The thermal pack 240 of FIGS. 30 and 31 can be configured in any of the ways disclosed in FIGS. 20, 23 and 25. However, in the thermal pack 240 of FIG. 30, the bendable and deformable support frame 166 is centrally located in the pouch 104. Furthermore, although the thermal pack 240 can be configured with connectors 102 for connecting the same to other similarly configured thermal packs, such connectors are not shown in FIG. 30. The thermal pack 240 of FIG. 30 includes one or more projections 242 extending from one or both ends of the thermal pack (FIG. 30 illustrates such projections 242 extending from both ends). The projections 242 include an adhesive for applying the thermal pack to a body part, such as a head of an infant, as shown in FIG. 31. The adhesive can be single or multiple use as is known in the art and can include a backing material that is peeled away to expose the adhesive for use.

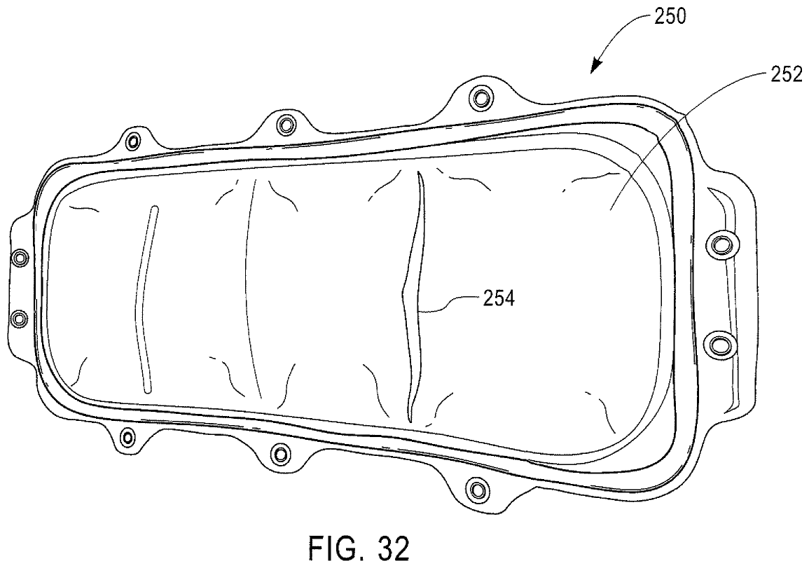

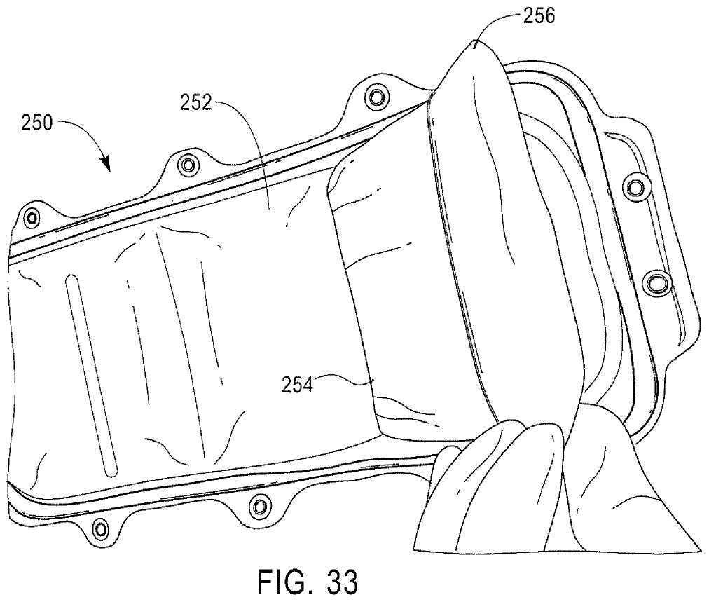

Turning next to FIGS. 32 and 33, there is illustrated another embodiment of a thermal pack, generally referred to by reference numeral 250. The thermal pack 250 can be configured as discussed above with regard to any of the above embodiments. However, the pouch 252 (a first pouch) of thermal pack 250 includes a slot 254 for accessing an interior of the pouch 252. The pouch 252 contains a second pouch 256 having the thermal material therein that can be inserted and/or removed through the slot 254. The second pouch 256 can be stored in a freezer or heated in a microwave and then inserted into the pouch 252 while in the cold or heated state.

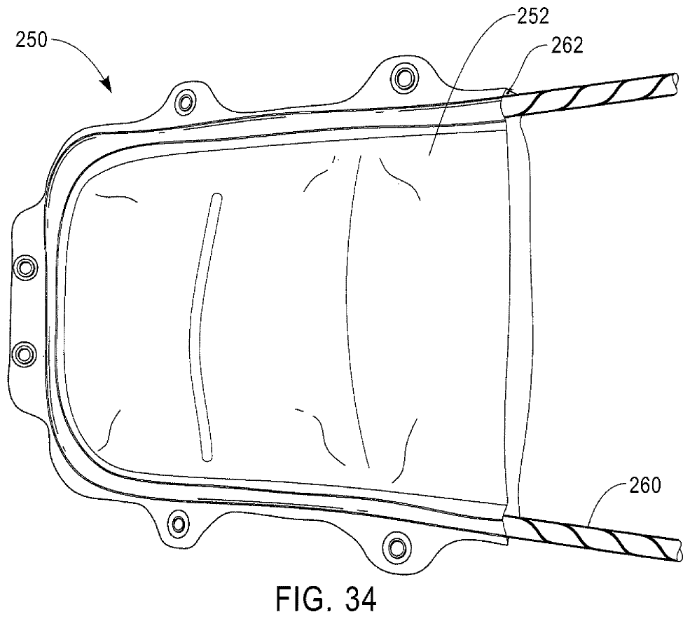

Turning now to FIGS. 34-36, the same illustrate an embodiment of a support frame/wire, generally referred to by reference number 260. FIG. 34 shows the support frame 260 for use with the thermal pack of FIG. 32, however, the support frame 260 can be used with any of the embodiments disclosed above, including that of FIG. 29. Although the support frame 260 is illustrated extending on all four sides of the pouch 252 in FIG. 34, the support frame 260 can extend on one, two or three sides. Furthermore, although the support frame 260 is shown being disposed in a cavity formed along the edges of the thermal pack 250, the support frame 260 can be releasable attached to the thermal pack 250, for example, as shown in FIG. 29.

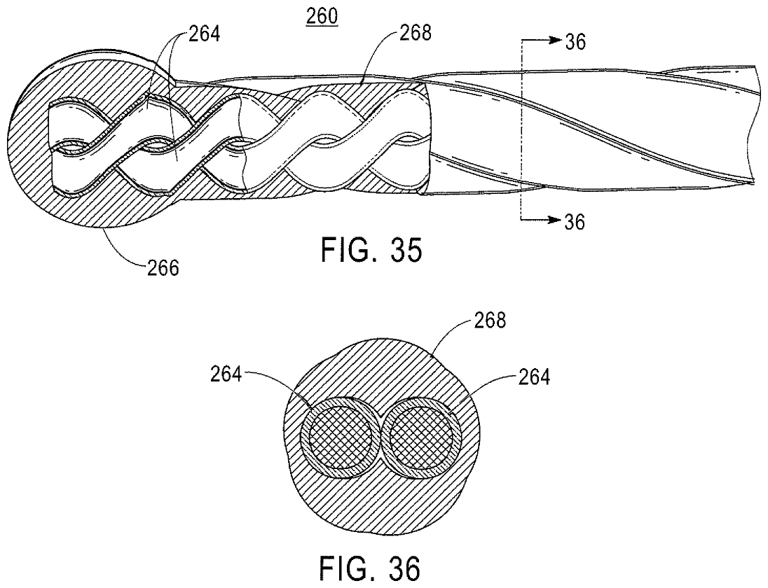

As shown in FIGS. 35 and 36, the support frame/support wire can comprise one wire or a plurality of twisted wires 264 extending along the length of the support frame 260. The wire or plurality of twisted wires 264 are then encased in a resin or elastomer, such as a foam, such as a silicone foam material 268 into a predetermined shape. Such shape can have an elongated shape, as shown in FIG. 35. Such shape can also include a bulb 266 at an end thereof to prevent ends of the wires 264 from puncturing the material of the pouch 252 or cavity 262.

Referring now to FIGS. 37-39, there is shown another embodiment of a thermal pack 300 having a segmented pouch body 302 formed of a plurality of pouch segments 304. The pouch segments 304 can be any shape or size and provided in any number, such as having a cube shape or a truncated cube shape where a depth of the pouch segments 304 is less than a length and width (cross-sectional shape) of the opening of the pouch segment. The thermal pack 300 of FIGS. 37-39 illustrate a great number of small pouch segments 304 for increasing flexibility of the pouch and concentrating an amount of thermal material contained in each pouch segment 304. As shown in FIG. 38, the segmented pouch body can be formed of a flexible material so as to conform to a body shape upon which it is applied, such as silicone rubber. The plurality of pouch segments for a cube or truncated cube cavity in which water or a separate cube can be disposed (as discussed below).

The thermal pack 300 can also have hook and loop (Velcro) straps 306 disposed at one end to a corresponding slot 308 which can be fastened to another end through corresponding slots 310. When the thermal pack 300 is wrapped around a body part, a free end of the hook and loop straps 306 can be inserted into the slot 310 and then fastened upon itself to fix the thermal pack 300 to the body part.

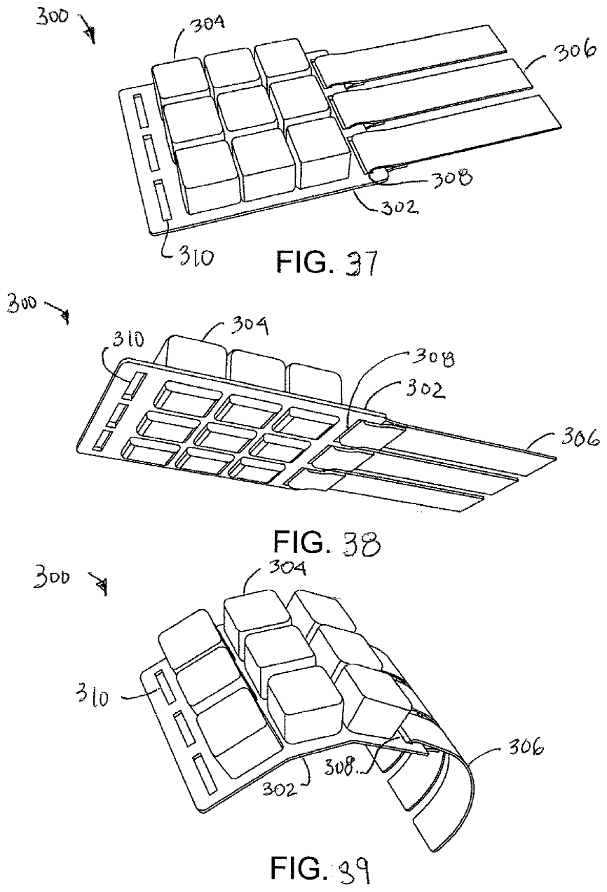

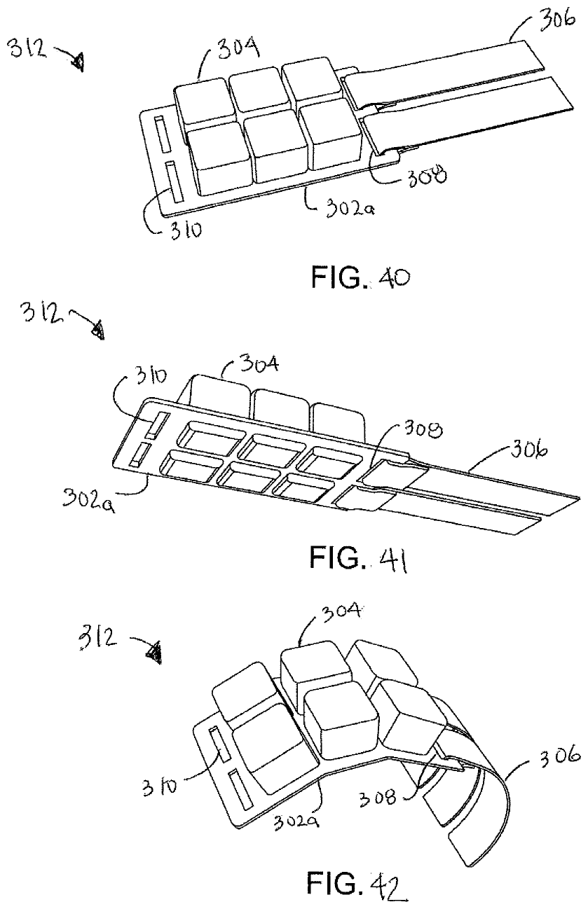

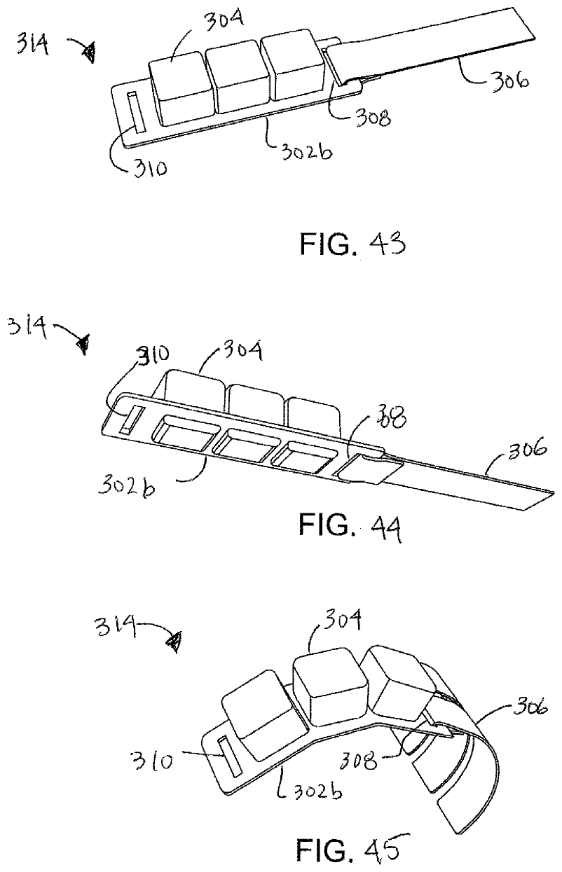

Although the thermal pack 300 is shown in FIGS. 37-39 having a segmented pouch body 302a having a 3×3 array of pouch segments 304, any number of rows and/or columns are possible. FIGS. 40-42 show a thermal pack 312 having a 3×2 array of pouch segments 304 with two hook and loop straps 306 corresponding to the two rows of pouch segments 304. Similarly, FIGS. 43-45 show a thermal pack 314 having a segmented pouch body 302b having a 3×1 array of pouch segments 304 with a single hook and loop strap 306 corresponding to the single rows of pouch segments 304.

Although FIGS. 37-45 show a hook and loop strap 306 corresponding to each row of pouch segments 304, more or less than the number of pouch segments 304 is possible. Although FIGS. 37-45 show the segmented pouch body 302, 302a, 302b having 3 columns of pouch segments 304, any number more or less than 3 is possible.

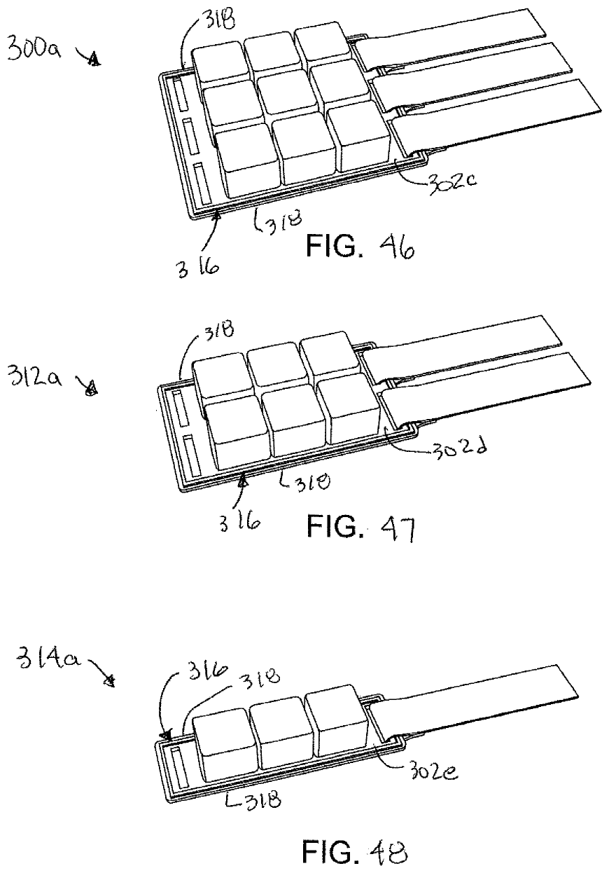

FIGS. 46-48 show the thermal packs 300, 312 and 314 of FIGS. 37, 40 and 43, respectively, also having a bendable and deformable support frame 316, such thermal packs being generally referred to as 300a, 312a and 314a, respectively. The bendable and deformable support frame 316 can be provided at all four sides of the pouch body 302c, 302d, 302e to outline the plurality of pouch segments 304 or on at least one or two opposing long sides 318 of the pouch body 302c, 302d, 302e, such as the two longer sides.

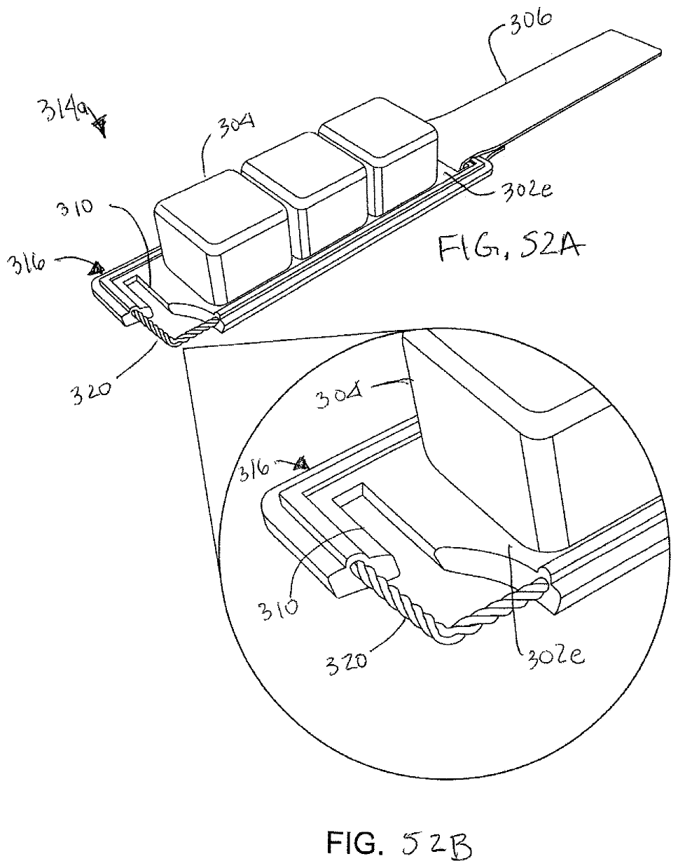

As shown in general with regard to the configuration of FIG. 48, FIGS. 52A and 52B illustrate the bendable and deformable support frame 316 having a pair of twisted wires 320 which can be embedded in a portion of the edge of the pouch body 302c, 302d, 302e, such as being insert molded therein. The twisted wires 320, can be at least a pair or more and can be formed of a resilient material, such as a ductile metal, that allows the pouch body 302c, 302d, 302e to be bent to conform to a corresponding shape of a body part and to maintain such shape until bent into another shape. Such resilient material should be able to withstand multiple changes in shape, such as avoid work hardening and becoming brittle. Embedding the twisted wires 320 in the portion of the edge of the pouch body 302c, 302d, 302e can prevent the twisted wires 320 from bending to an extreme angle which may cause such work hardening. Although shown with a pair of helically twisted wires, the wires can be provided in two or more wires and can be twisted in another configuration or a single wire may be used as the bendable and deformable support frame 316.

During use, as shown in FIG. 49, the cavity 304a defined by the pouch segments 304 can be filled with a liquid capable of freezing, such as water, and frozen prior to applying the thermal pack to a body part. Although the embodiment of FIG. 37 is shown in FIG. 49, any of the similar embodiments disclosed herein can be used in the same manner.

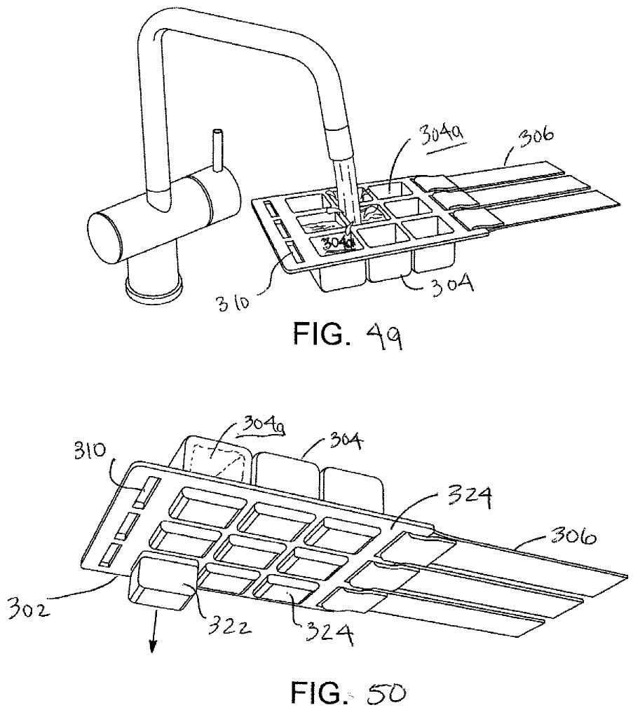

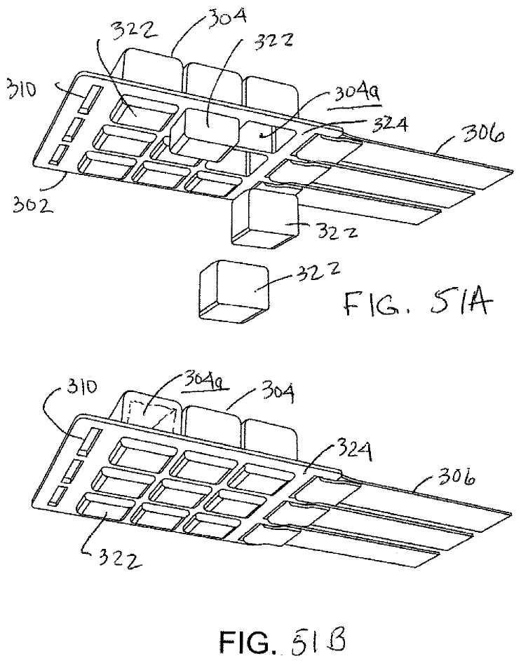

Referring now to FIGS. 50, 51A and 51B, there is shown another use for the thermal packs shown in FIGS. 37-48 where the cavities 304a defined by the pouch segments 304 have cubes 322 disposed therein that are themselves capable of being frozen. Such cubes 322 can have a similar length and width as the cavities 304a (i.e., the cross-section shape of the cavity) and either have a similar depth as the cavities 304a so as to have a surface of the cubes 322 being substantially flush with a lower surface 324 of the pouch body (302 in FIGS. 50, 51A and 51B) or a larger depth than the cavities 304 such that the cubes 322 extend from the cavities 304a and from the lower surface 324. Although FIGS. 50, 51A and 51B illustrate the embodiment of FIG. 37, any other of the similar embodiments discussed above can be used with the cubes 322.

The cubes 322 can be formed of stainless steel and have an internal cavity having a substance, such as water or a gel, or a water and glycerol solution. Such stainless steel ice cubes are well known in the art, for example, those used to keep alcoholic drinks colder than ice while not diluting the same when the ice melts. Such cubes 322 can stay colder longer than ice and are not as messy as filling with water or other liquids.

Furthermore, although the pouch segments are illustrated as being a cube or partial cube shape, any other shapes can be used, such as a partial sphere. Additionally, although the cubes are also shown as being a cube shape, their shapes can match, at least partially, the shape of the pouch segments. Where the cubes are spherical, the pouch segment can have a partial spherical shape, which can be more than half of a sphere such that the pouch segment can deform to allow the spherical cube to be inserted, in which case, the pouch segments can return to their shape after the cube is inserted, thereby also retaining the spherical cube in the pouch segments.

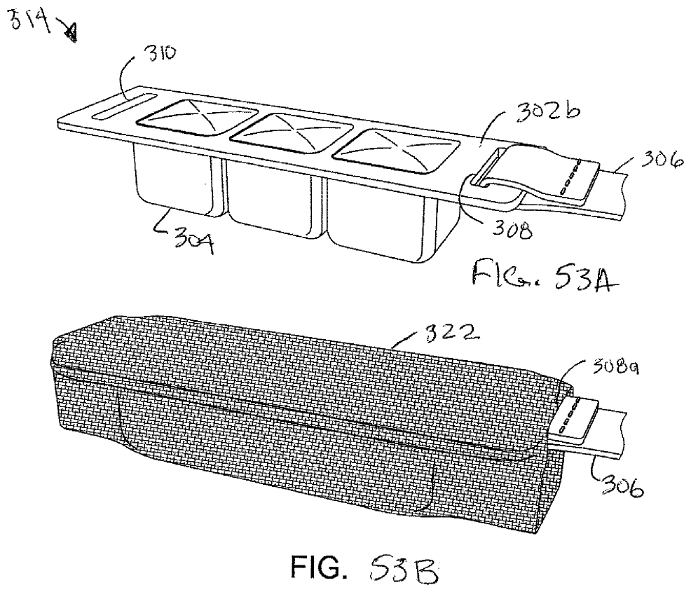

Turning next to FIGS. 53A and 53B, any of the embodiments discussed above having the pouch segments 304, either when filled with a liquid, such as water and frozen or when filled with the cubes 322 can be used “naked” as shown in FIG. 53A or with a corresponding cover 326 as shown in FIG. 53B. The cover 326 can be sized to fit the corresponding pouch body 302b including a corresponding hole 308a for the hook and loop strap 306 to extend from the cover 326. Although the cover can also have a hole corresponding to the slot 310, the cover 322 can be formed or have a portion configured to latch to the end of the hook and loop strap 306. Furthermore, the cover 322 can have a flap with hook and loop material on opposing surfaces of the flap and cover 322 for securing the cover on the thermal pack 314 once the thermal pack is secured within the cover. The cover can be any natural or synthetic woven fabric. Although the thermal pack in FIGS. 53A and 53B is shown as having a similar configuration as the thermal pack 314 shown in FIG. 45, the cover can be configured for any configuration of thermal pack having any number of the pouch segments 304.

Since many modifications, variations, and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A thermal pack comprising:
a flexible pouch body having a plurality of pouch segments each defining a cavity;
and one or more hook and loop straps attached to a first end of the pouch body, where the pouch body has an opening for accepting a second end of the hook and loop strap for securing the pouch body to a body part.

2. The thermal pack of claim 1, wherein the pouch segments are formed in an array of the plurality of pouch segments.

3. The thermal pack of claim 1, wherein each cavity has a cross-sectional shape with a length and width having an equal dimension and having a depth less than the equal dimension.

4. The thermal pack of claim 1, further comprising a bendable and deformable support frame having at least two twisted wires on at least two longer sides of the pouch body.

5. The thermal pack of claim 4, wherein the at least two twisted wires are embedded in a portion of an edge of the pouch body.

6. The thermal pack of claim 4, wherein at least two twisted wires are formed of a ductile metal.

7. The thermal pack of claim 1, further comprising a cover for covering portions of the pouch body.

8. The thermal pack of claim 7, wherein the cover covers all portions of the pouch body.

9. The thermal pack of claim 7, wherein the cover is formed of a woven fabric.

10. The thermal pack of claim 1, further comprising metallic cubes disposed in each of the cavities of the pouch segments, each of the metallic cubes having a metallic shell defining a cavity with a freezable substance.
